(19) United States
(12) Patent Application Publication
(10) Pub. No.: US 2003/0148265 A1
(43) Pub. Date: Aug. 7, 2003

(54) Anti-viral conjugate comprising a factor allowing the translocation of a protein across a cell membrane and comprising a single-chain antibody fragment directed against a viral protein

(76) Inventors: Timothy John Gilby Brooks, Wiltshire (GB); Jacqueline Marie Duggan, Wiltshire (GB)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/204,007

(22) PCT Filed: Feb. 14, 2001
(86) PCT No.: PCT/GB01/00586

(30) Foreign Application Priority Data
Feb. 15, 2000 (GB) 0003284.7

(51) Int. Cl. C12Q 1/70; A61K 39/42; C12N 7/00; C12N 1/21; C12P 21/02; C07K 16/08

(52) U.S. Cl. 435/5; 530/388.3; 424/159.1; 435/235.1; 435/69.1; 435/320.1; 435/252.3

(57) ABSTRACT

A protein conjugate comprising conjugate comprising a first region comprising a factor that permits translocation of a protein across a cell membrane; and a second region comprising a single-chain antibody fragment which has affinity for a viral protein, in particular a viral protein which is necessary for replication of a virus such as a flavivirus.

### FIELD OF THE INVENTION

The present invention relates to the treatment of viral infections. In particular, the present invention relates to conjugates such as protein conjugates or polynucleotides encoding these for the treatment of infection by viruses such as viruses of the Flaviviridae family as well as alphaviruses.

### BACKGROUND OF THE INVENTION

There is a great need for effective antiviral therapies against a wide variety of viruses.

For example, Hepatitis C virus (HCV) is a major cause of morbidity world-wide, and leads to a high incidence of persistent disease. It is responsible for the majority of cases of non-A non-B hepatitis (NANBH) in the West, and is spread by blood transfusion. The majority of people infected develop chronic hepatitis and many of these go on to develop cirrhosis of the liver. The infection can persist for many years and the virus is also known to play a role in hepatocellular carcinoma.

HCV belongs to the Flaviviridae family of viruses, which also includes dengue virus and tick-borne encephalitis virus (TBE). The Flavivirus group share a common genomic structure and the RNA genome is single stranded and of positive polarity. The RNA genome encodes the necessary enzymes for RNA replication. The coding sequence for the mature proteins may be represented as:

NH₂-[C-prM-E1-E2/NS1-NS2-NS3-NS4A-NS4B-NS5A-NS5B]-COOH

Protein C is the structural core protein, E1 and E2/NS1 are the envelope proteins and the remaining proteins are non-structural proteins associated with the replicative process.

Of particular interest from an anti-viral viewpoint is the NS3 protein which has three main functions: a serine protease, a nucleoside triphosphatase (NTPase) and a helicase function. The protein is cleaved from NS2 by the combined action of the proteolytic activities of NS2 and NS3, and then performs other virally-encoded proteolytic reactions on its own. These functions appear to be conserved across the Flavivirus group and this has led to much interest in the protein as a target for anti-viral therapy. However, the design of small molecule inhibitors has proved difficult. So far, only interferon alpha (IFN-α) and interferon beta (IFN-β) have been used to treat HCV infection. The success rate in persistent cases is about 30% and the remaining patients are non-responsive to IFN treatment.

Alternative vaccines are under development for HCV and generally consist of recombinant analogues of the putative viral structural proteins (C, E1, E2). However, different viruses with different envelope proteins are able to evade the immunological challenge, and so effective treatment may not be available.

Therefore, while some treatments are available, there is a very real need for effective alternative treatments for Flavivirus infection.

Another family of viruses which are of clinical significance is the Alphavirus genus of the family Togaviridae, which are small, enveloped viruses with a positive sense RNA genome. The structural proteins of the alphaviruses are translated from a 26s RNA. The genes encoding these proteins are contained within a single open reading frame in the order:

H₂N-[nsP1-nsP2-nsP3-nsP4-capsid-E3-E2-6K-E1]-COOH

Venezuelan equine encephalomyelitis virus (VEEV) is a member of this genus and was first described by Kubes and Rios in 1939 (Kubes and Rios, (1939) Science, 90, 20-21.). It causes epidemic and endemic disease in the Americas. Outbreaks of epidemic disease are a major health and economic problem in Central and South America (Johnson, K. M. and Martin, D. M., (1974). Venezuelan equine encephalitis. In Advances in Veterinary Science and Comparative Medicine, eds. Brandley, C. A. and Cornelius, C. E., Academic Press, New York and London, pp. 79-116.). During epidemics, millions of horses can be affected with a fatality rate up to 80%. Although endemic strains are of less importance economically (they do not cause encephalitic disease in equines), all VEE strains cause debilitating disease in humans with a fatality rate of about 1%. The increased incidence of travel and changes in agricultural and irrigation practices in developing countries in the Americas may lead to an increased incidence of endemic disease amongst exposed humans. Epidemic disease was thought to be extinct until an epidemic outbreak in 1992-1993, caused by a IC subtype virus. Another epidemic in Venezuela and Colombia in 1995 caused hundreds of thousands of disease cases amongst horses and humans (Rivas et al., 1995, J. Infect. Dis. 175, 828-832).

Although antibodies and antibody fragments are known which inhibit a number of viruses by inhibiting viral enzymes, delivery of these to cells is required before a clinical effect can be achieved. Previous approaches to this problem have focussed on intracellular expression of the antibodies (Takekoshi et al, 1998, J. Virol.Methods, 74, 89-98, M. BouHamdan et al., Gene Therapy (1999), 6, 660-666) or using virus vectors, such as Sindbis virus (Jiang et al, J. Virol. (1995) 69, 1044-1049). However, such approaches have not proved to be particularly useful in practice. Use of a virus for intracellular immunisation would elicit a strong immune response, which may impact on the efficacy of the antibody.

Conjugates that contain the homeodomain of Antennapedia are described in WO99/11809. This homeodomain is used to translocate proteins into cells for a variety of therapeutic purposes, including antiviral purposes. It has been suggested that recombinant antibodies may be used in this way.

The applicants have found however that full size antibodies, or even full length single chains derived from antibodies are too large to be effectively transported in this way.

### SUMMARY OF THE INVENTION

The present invention is based on the realisation that viral infection, such as alphavirus or flavivirus infection and particularly Flavivirus infection, may be treated with a single-chain antibody fragment that inhibits a viral protein, in particular a protein necessary for viral replication, and that a suitable delivery system can be constructed using a cell membrane translocation factor to transport the agent across a cell membrane to target the viral protein.

According to the present invention, there is provided a protein conjugate comprising a first region comprising a factor that permits translocation of a polypeptide across a cell membrane and a second region comprising a single-chain antibody fragment (scFv) having affinity for a viral protein.

As used herein, the term “polypeptide” encompasses large peptides such as proteins. The expression “protein conjugate” includes complexes and fusions of polypeptides or proteins.

Single-chain antibody fragments (scFv) consist of the variable light and heavy chain regions of an antibody, suitably a monoclonal antibody, that are joined together by a short peptide linker designed to allow conformational folding of the variable regions to form the antigen binding site. They were first described in 1990 (McCafferty et al, Nature, 348, 552-554, 1990).

ScFvs can be expressed in a variety of different expression systems, such as bacteria, viruses, yeast and plants. ScFvs can be expressed as phage fusion proteins and large phage libraries expressing scFvs with different specificities can be produced. ScFvs with desired binding characteristics can be selected from the phage display libraries by panning against an viral protein of choice.

They are particularly appealing as therapeutic agents due to their small size (generally up to about 300 amino acids long) and short half-life.

The scFv used in the construct of the invention may be specific for a target protein derived from any virus. Examples of such viruses include flaviviruses, such as hepatitis C, dengue virus and tick-borne encephalitis virue (TBE), alphaviruses as discussed above as well as enteroviruses, arboviruses, retroviruses such as immunodeficiency viruses like HIV, respiratory viruses such as the influenza group viruses, rhabdoviruses such as rabies, herpes viruses, human papilloma virus (HPV), adenoviruses, adenaviruses such as hepadenavirus (hepatitis B) and pox viruses. In particular, the virus will comprise a Flavivirus, e.g. hepatitis C virus, dengue virus or tick-borne encephalitis virus. Selection of suitable scFvs can be made on the basis of the literature in some cases, or using conventional methods such as the phage library screening method described above.

The polypeptide of said second region comprises an scFv that has affinity for a viral protein which may be a non-structural (NS) protein or a structural protein such as an envelope protein. Preferably, the second region is a scFv which binds a protein necessary for the replication of a virus

The scFv is suitably able to inhibit a viral protein which is essential for replication. In the case of Flaviviruses, these are suitably the non-structural proteins such as those identified as NS1, NS2, NS3, NS4A, NS4B, NS5A and NS5B and suitably NS2, NS3, NS4A, NS4B, NS5A and NS5B. In particular, the proteins targeted are NS2 or NS3 proteins and preferably NS3 proteins. Suitable alphavirus proteins which are targeted by the constructs of the invention are the nsP1, nsP2, E1 and/or E2 proteins.

In a preferred embodiment of the invention, the translocation factor comprises the homeodomain of antennapedia, or a functional fragment thereof. Typically, the translocation factor or the whole protein conjugate will be non-denatured, i.e. prepared under conditions which do not disrupt intramolecular bonds.

Alternatively, the translocation factor may be expressed recombinantly from an expression host such as a prokaryotic or eukaryotic host cell, preferably a prokaryotic cell such as E. coli. Depending upon the host cell, however, the protein product may require refolding before it may be used in say a mammalian system, and this can be determined using routine methods.

For example, when expressed in a prokarytic system such as E. coli, refolding in a refolding buffer such as an arginine refolding buffer has been found to be advantageous to allow efficient translocation of the factor into a mammalian cell.

As discussed above, the second region may specifically bind the protein necessary for the replication of the virus such as the Flavivirus such that in inhibits or inactivates the activity of said protein.

Alternatively, it may act as a “targeting” mechanism for one or more therapeutic agents such as inhibitors of the serine protease, NTPase or helicase functions of viral proteins such as NS3. In this case, the conjugate further comprises or is associated with the additional therapeutic agent.

In a preferred embodiment, the conjugate of the invention comprises a fusion of the translocation factor, the polypeptide having affinity for the viral protein, and optionally also the therapeutic agent.

Further targeting of the active regions may be achieved by including further intracellular localization or targeting moieties into the conjugate. An example of such a moiety is the ANTP protein. Such moieties may be directly attached to or associated with the second region and/or the therapeutic agent that it is intended to localize within the cell.

All these various regions and moieties may be directly adjoining each other or they may be spaced apart by means of spacer amino acid sequences.

If required, cleavage sites may be introduced in said spacer regions. These cleavage sites may be short sequences which are the target for intracellular protease enzymes. In this way, the regions may be separated after transport into the cell so as to prevent inhibition of the effect of the second region and/or the therapeutic agent(s).

Fusion proteins of this type may be expressed from a single polynucleotide, and these form a further aspect of the invention.

These polynucleotides may be incorporated into an expression or replication vector as are well known in the art and used to transform organisms such as cells (prokaryotic or eukaryotic) and viruses. Such vectors and organisms form yet further aspects of the invention.

The conjugates described above have useful therapeutic value in that they can penetrate an infected cell and deliver an antibody into the cell to target an essential protein of viral replication, to inhibit replication. The conjugate may be administered per se to an individual in need thereof, or a polynucleotide encoding it may be administered in a form in which it is expressed in the host system.

Thus in a further aspect of the invention there is provided a pharmaceutical composition comprising a conjugate as described above, a polynucleotide which encodes a fusion protein as described above, or a recombinant organism such as a microorganism or a virus which comprises said polynucleotide, and which expresses said protein conjugate, in combination with a pharmaceutically acceptable carrier or diluent.

Particular cells which are useful for therapeutic purposes include gut-colonising organisms which are preferably attenuated, such as attenuated Salmonella. Suitable recombinant viruses which can act as carriers for the protein conjugate of the invention are attenuated viruses, for example attenuated vaccinia viruses.

Where protein conjugate is required for therapeutic use, it is suitably obtained by expression of a fusion protein in a suitable recombinant cell. Thus, according to a further aspect embodiment of the invention, a host cell is transformed or transfected with a polynucleotide that encodes a protein conjugate, as described above, in the form of a fusion protein. The host cell may be used in the preparation of the proteins. Typically, the proteins of the present invention will be purified from a host cell under non-denaturing conditions, or may be subjected to refolding subsequent to purification, as exemplified hereinafter. If necessary, the preparation can be carried out in the presence of protease inhibitors.

The present invention provides a means to overcome the general difficulty of delivering relatively large proteins such as antibodies to an intracellular site where the virus replicates.

### DESCRIPTION OF THE INVENTION

The invention will now be further described by way of illustration only.

In one particular embodiment, the invention comprises a protein conjugate having affinity for a protein such as the NS3 protein of a Flavivirus.

In one particular embodiment, the invention comprises a protein conjugate having affinity for the nsP1, nsP2, E1 or E2 protein of an alphavirus.

A protein conjugate according to the invention is typically a fusion protein comprised of two distinct regions. The first region comprises the translocation factor to transport the protein across the cell membrane. Typically, the translocation factor will be a protein that corresponds to the DNA-binding region of the Drosophila and antennapedia homeoprotein (Schutze-Redelmeier et al, J. Immunol. (1996) 157:650-655). The homeodomain of the antennapedia molecule spontaneously crosses cellular membranes and has been used previously to deliver small antigenic peptides into a cell. The homeodomain comprises 60 amino acids, although subsequent work has shown that a truncated version comprising only 16 amino acids can translocate across a cell membrane (Prochiantz, Current Opinion in Neurobiology (1996) 6:629-634). Therefore, the present invention encompasses the use of the complete homeoprotein, or a functional fragment thereof.

The homeodomain is conserved in many different organisms and therefore functional homologues are also envisaged for use in the present invention. Typically, a homologue will have >60%, preferably >80% sequence homology to the homeodomain of Drosophila or antennapedia.

As used herein, the term “sequence homology” refers to levels of protein similarity which may be determined by for example using known algorithms such as that the multiple alignment method described by Lipman and Pearson, (Lipman, D. J. & Pearson, W. R. (1985) Rapid and Sensitive Protein Similarity Searches, Science, vol 227, pp1435-1441). The “optimised” percentage score should be calculated with the following parameters for the Lipman-Pearson algorithm:ktup=1, gap penalty=4 and gap penalty length=12. The sequence for which similarity is to be assessed should be used as the “test sequence” which means that the base sequence for the comparison should be entered first into the algorithm. Generally, homeodomain of the Drosophila or antennapedia or a functional fragment thereof, will be used as the reference sequence.

The ability of the homeoprotein to transport a cargo across the cell membrane may be dependent on retaining its native structure. It may therefore be important to prepare or purify the protein under non-denaturing conditions. The importance of this requirement is disclosed in International Patent Publication No. WO-A-9911809.

Alternative translocation factors may also be used such as the tat protein from HIV or the herpes simplex virus type I tegument protein VP22 or a functional fragment or homologue thereof (see for example WO 98/32866).

The protein conjugate of the present invention also comprises a second region which specifically binds or inhibits expression of a target viral protein and so displays antiviral activity. The second region will comprise a single-chain Fv fragment which includes at least part of the variable region so as to confer affinity for the target protein, such as the NS3 protein or E1 protein.

Single-chain antibody fragments used in the invention may be derived from antibodies with the desired activity in a conventional manner. Antibodies having an affinity for the target proteins may be obtained using various techniques. For example, the antibody may be produced by classical hybridoma technology, comprising the fusion of B-lymphocytes from immunised animals with an appropriate fusion partner. Alternatively, mRNA may be purified from selected lymphocytes and amplified using the technique of PCR. Phage display libraries may also be used.

A preferred embodiment is the use of single-chain antibodies (ScFv) which retain affinity for NS3 protein. Typically, single-chain antibodies comprise less than 300 amino acids and are therefore suitable to be transported using the translocation factor. Typically, the antibody or fragment has affinity for the target protein such as the NS3 protein of greater than 10⁵ l/mol, preferably greater than 10⁸ l/mol and most preferably greater than 10¹⁰ l/mol.

Viral proteins which may be used to raise the antibodies are either known or may be isolated from the target virus using conventional methods. For example, NS3 proteins which may be used for raising an immune response are known from International Patent Publication No. WO-A-9727334 which discloses procedures for the expression and purification of an enzymologically active NS3 protein.

A protein conjugate according to the present invention may be prepared using any suitable means. For example, the protein conjugate may be a fusion protein, where a polynucleotide encoding the translocation factor and a polynucleotide encoding the scFv are fused in-frame using recombinant DNA technology and transformed or transfected into a host cell which then encodes the protein. The host cell may be any suitable protearyotic or eukaryotic cell.

Typically E. coli is used. The polynucleotide that encodes the protein conjugate may also comprise suitable expression control sequences, e.g. promoters. Suitable control sequences will be apparent to the skilled person. Suitable methods for producing the fusion proteins are disclosed in International Patent Publication No. WO-A-9911809.

Alternatively, the protein conjugates may be prepared by linking the two regions chemically, e.g. via a thiol linker. A method for preparing conjugates with thiol linkers is disclosed in Theodore et al, J. Neurosci. (1995) 15(11):7158-7167.

On binding to the viral protein such as the NS3 protein, the antibody of the protein conjugate may react to inhibit the function of the viral protein to prevent virus replication. Alternatively, the antibody may target a further therapeutic agent with which it is associated or bonded, to the viral protein leading to inactivation. A suitable therapeutic, where the target protein is the NS3 protein of Flavivirus may be an agent that inhibits the serine protease activity of the NS3 protein.

Alternatively, the NTPase and the helicase function of the viral protein may be inhibited. Other suitable therapeutic agents will be apparent to the skilled person in the art.

The protein conjugates, polynucleotides or carriers therefore according to the present invention may be formulated with any suitable excipient or diluent. These may be solid or liquid and will vary depending upon the nature of the entity being administered. Gut-colonising organisms such as attenuated Salmonella strains for example, will be administered orally. Viral delivery systems or “naked DNA” type therapies, as well as treatment with the protein itself, may be better achieved by formulations which are intended for parenteral administration. Typically, the conjugate proteins will be administered intravenously, and formulations suitable for this will be apparent to the skilled person.

In the case of localised viral infections, such as Herpes virus infections, or papilloma virus infections, administration topically may be preferred, and suitable formulations include creams for topical administration. Alternatively administration by local injection may be useful.

The amount of protein that is required for the treatment of the viral infection will depend, at least in part, on the affinity level of the antibody for the viral protein, the means for administering the agent, the severity of the disease state, the nature of the patient etc. and will be determined using clinical practice.

A further aspect of the invention comprises a method of treating a patient suffering from a viral infection, which method comprises administering to said patient, a conjugate as described above.

The invention further comprises a conjugate as described above for use in antiviral therapy.

Yet a further aspect comprises a conjugate as described above for use in the preparation of a medicament for the treatment of viral infection.

The invention will be described by way of Example with reference to the accompanying diagrammatic drawings in which:

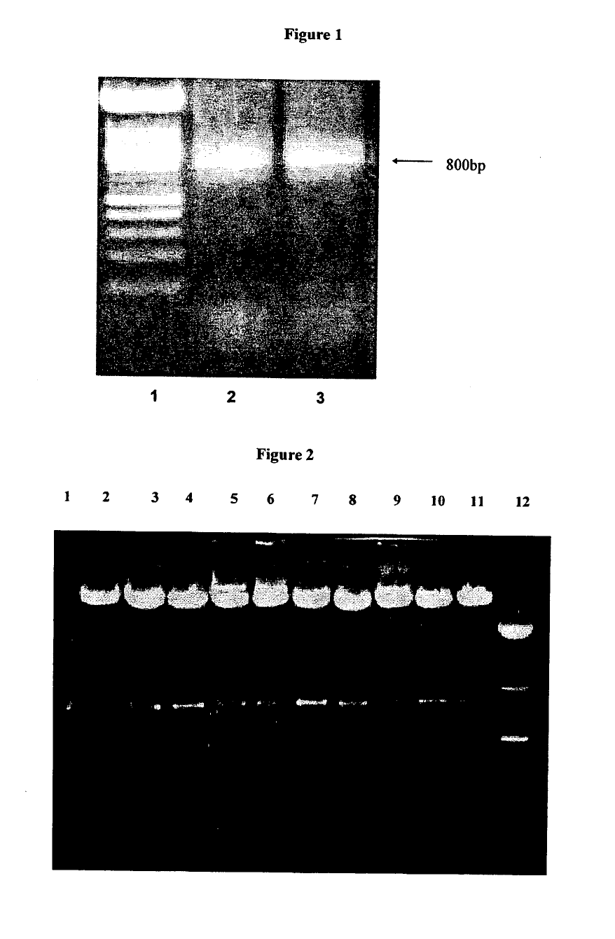

FIG. 1 shows a gel photograph of the PCR amplified scFv DNA in which Lane 1=Molecular weight marker (Boehringer Mannheim MWM III) and Lanes 2 and 3=H10 scFv DNA;

FIG. 2 shows a restriction digestion of pET6-H10 clones, where Lanes 1 and 12=100 bp DNA ladder; and Lanes 2-11=Restriction digestion products of nine pET6-H10 clones:

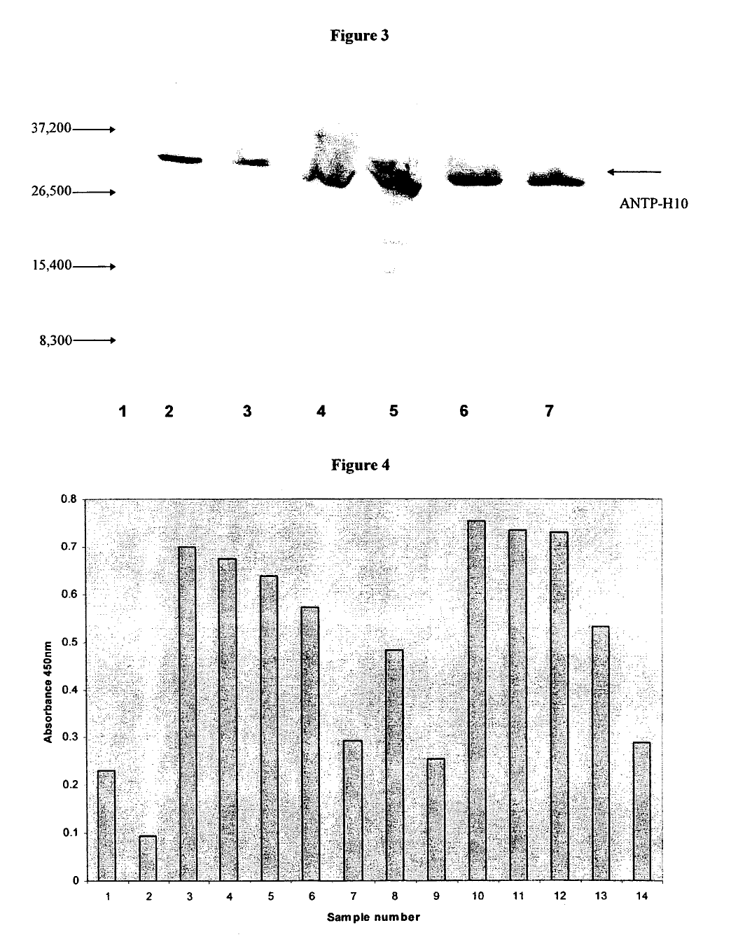

FIG. 3 is a western blot of soluble, insoluble and re-solubilised ANTP-H10, where Lane 1=Molecular weight marker; Lane 2=soluble ANTP-H10; Lane 3=soluble ANTP-H10; Lane 4=insoluble ANTP-H10; Lane 5=insoluble ANTP-H10; Lane 6=re-solubilised ANTP-H10; Lane 7=re-solubilised ANTP-H10:

FIG. 4 is an ELISA of soluble and re-solubilised ANTP-H10 after purification where Sample 1=soluble fraction flow-through; Sample 2=soluble fraction wash buffer; Samples 3-7=soluble fraction eluted samples 1-5; Sample 8=re-solubilised fraction flow-though; Sample 9=re-solubilised fraction wash buffer; and Samples 10-14=re-solubilised fraction eluted samples 1-5:

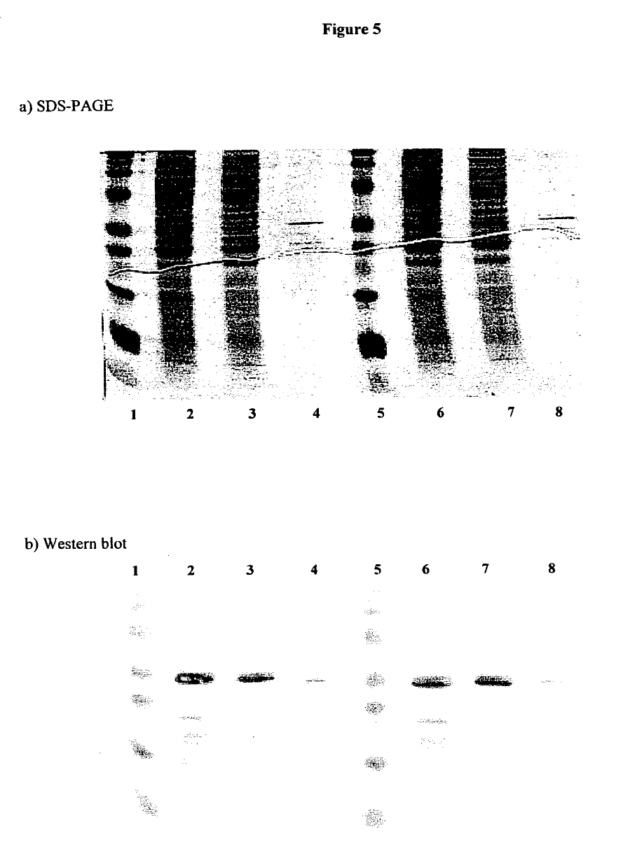

FIG. 5 is an SDS-PAGE(5a) and Western blot (5b) of soluble and re-solubilised ANTP-H10, where Lanes 1 and 5=molecular weight marker, Lane 2=soluble fraction flow-through, lane 3=soluble fraction wash buffer, lane 4=purified ANTP-H10 from soluble fraction, lane 6=re-solubilised fraction flow-through, lane 7=re-solubilised fraction wash buffer, lane 8=purified ANTP-H10 from re-solubilised fraction; and

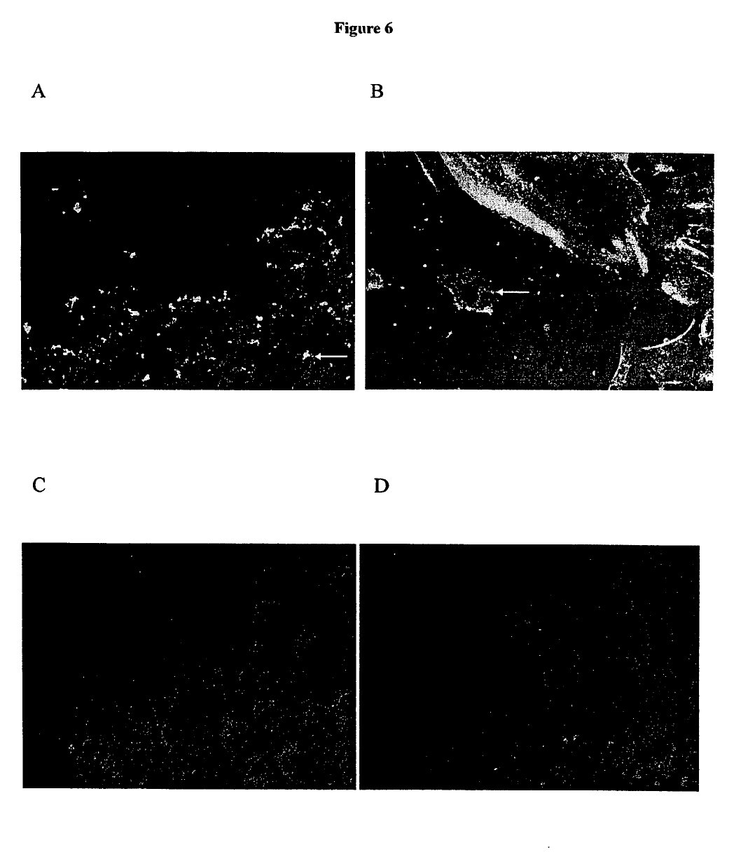

FIG. 6 illustrates the translocation of ANTP-H10 re-folded in arginine and TN buffer into Vero cells, where A=translocation of ANTP-H10 re-folded in TN buffer, B=translocation of ANTP-H10 re-folded in arginine buffer, and C and D=cell only controls with no ANTP-H10 added to the cells.

### EXAMPLE 1

Cell Lines and Bacterial Strains

Vero cells (African green monkey kidney cell line, obtained from ECACC, no. 84113001) were maintained in Glasgow minimal essential medium containing 10% (v/v) foetal calf serum (FCS), 1% (v/v) L-glutamine and 1% (v/v) penicillin/streptomycin (all purchased from Sigma Chemical Co., Poole, Dorset).

Chemically competent E. coli strain DH5α was purchased from Gibco BRL, Paisley, Scotland. Chemically competent E. coli strain BL21-Gold (DE3) pLysS was purchased from Stratagene.

Cloning H10 scFv into pET6-Paolo

pET6-Paolo was obtained from Colin Ingham, Imperial College, London. The plasmid is derived from pET29b, obtained from Novagen, with the DNA encoding the 60aa homeodomain of Antennapedia cloned between the Nde I and Bam HI sites and a c-myc tag cloned between the Hind III and Xho I sites. VEE E1 specific scFv H10, derived from the hybridoma cell line MH2, cloned into a phage display vector pAK100 was PCR amplified from the phage display vector using the following primers:

| | |
|---|---|
| V_{L} primer | |
| 5′ CTGGCGAATTCATGGCGGACTACAAAG 3′ | (SEQ ID NO 1) |
| V_{H} primer | |
| 5′ GGAATTGAGCTCCGAGGAGAC 3′ | (SEQ ID NO 2) |

The underlined area under the V_{L} primer denotes an Eco RI restriction site. The ATG codon after the restriction site denotes the start of the scFv light chain sequence. The underlined area under the V_{H} primer denotes the SAC I restriction site. The sequence after the restriction site denotes the 3′ sequence of the end of the V_{H} region.

In this way, Eco RI and Sac I restriction sites for cloning into pET6-Paolo were introduced. The PCR was set up using 1 μl DNA, 1 μl 10 mM dNTPs (Boehringer Mannheim), 1 μl 1 mM MgSO₄, 0.5 μl each primer (100 pmol/μl, synthesised by Cruachem Ltd., Glasgow), 5 μl 10×PCR buffer 2 (EXPAND High Fidelity PCR system, Boehringer Mannheim). The PCR reaction was heated to 95° C. for 5 minutes and 2.5 units DNA polymerase (EXPAND High Fidelity PCR System) was added to the PCR reaction. The reaction was cycled for 7 cycles of 92° C. 1 min, 58° C. 50 secs, 63° C. 30 secs, 72° C. 1 min, followed by 23 cycles of 92° C. 1 min, 63° C. 1 min and 72° C. 1 min. The PCR reaction mix was run out on a 1.5% agarose gel. The amplified scFv DNA at ˜800 bp was excised and purified using a Bio 101 Geneclean kit according to the manufacturer's instructions.

The PCR amplified scFv DNA and plasmid pET6-Paolo were digested with Eco RI and Sac I for 2 hours each at 37° C. (the digested DNA samples were cleaned using a Bio 101 Geneclean kit between each digestion). 2 μl H10 scFv DNA was ligated into 2 μl pET6-Paolo using 1 μl T4 DNA ligase (Boehringer Mannheim) and 1 μl 1×ligation buffer in 10 μl total volume (made up with dH₂O), with incubation overnight (overnight) at 16° C.

The gel photograph showing the PCR amplified scFv insert is shown in FIG. 1. A band at ˜800 bp, which is the expected size of the scFv DNA, was obtained from the PCR.

Transformation into DH5α and Analysis of Clones

The scFv DNA was excised and purified, and digested with Eco RI and Sac I. The plasmid pET6-Paolo was also digested. H10 scFv DNA was ligated into pET6-Paolo and transformed into E. coli DH5α.

2 μl ligated pET6-H10 (pET6-Paolo containing H10 scFv DNA) was transformed into 100 μl competent DH5α using heat shock at 42° C. for 50 seconds. Transformed cells were recovered using 900 μl SOC medium (GibcoBRL) and incubating at 37° C. for 1 hour. 100 μl transformation reaction was plated out onto two L-agar plates containing 50 μg/ml kanamycin and incubated overnight at 37° C.

Ten colonies were picked and inoculated into 5 ml L-broth+50 μg/ml kanamycin. The cultures were grown overnight at 37° C. with shaking at 200 rpm. Glycerol stocks were made of each clone by adding 800 μl saturated culture to 200 μl 80% sterile glycerol and storing at −70° C. The remaining culture was centrifuged at 3000 g for 10 minutes to pellet the cells and the DNA was extracted and purified using a Qiagen plasmid mini-prep kit according to the manufacturer's instructions. Purified plasmid DNA was subjected to restriction digestion using Eco RI and Sac I to determine the presence of the scFv inserts in each clone and the DNA was sequenced at the N-terminus by Oswel Ltd., Southampton to determine the correct orientation of the insert in the vector and to ensure that the scFv DNA was cloned in-frame with the ANTP DNA.

FIG. 2 shows the gel photograph of the restriction digestion of the clones. The gel shows the result of the restriction digestion of pET6-H10 DNA with Eco RI and Sac I. All clones except clone 1 produced a DNA band at ˜800 bp, which is the expected size of the scFv insert, indicating that these clones contained an scFv insert. The lack of an insert for clone 1 suggests that this clone does not contain an scFv insert.

Transformation of pET6-H10 into BL21-Gold (DE3) pLysS

1 μl pET6-H10 clone 10 DNA was transformed into competent BL21-Gold (DE3) pLysS cells for expression of ANTP-H10 protein. The cells were heat-shocked at 42° C. and recovered using SOC medium as described previously. Ten clones were picked from the transformation plates and inoculated into 5 ml L-broth+50 μg/ml kanamycin. The cultures were grown overnight and a loopful of each overnight culture was plated out onto fresh L-broth+kanamycin plates. The plates were incubated overnight and stored at 4° C. The remaining culture was centrifuged at 3000 g for 10 minutes to pellet the cells and the plasmid DNA was extracted and purified using a Qiagen plasmid mini-prep kit. Each plasmid DNA was subjected to restriction digestion using Eco RI and Sac I to determine the presence of the scFv insert.

Clone 1 was chosen for the production of ANTP-H10 protein.

Growth of BL21 Containing pET6-H10 and Expression of ANTP-H10

One colony (Clone 1) of BL21 containing pET6-H10 was taken from an L-agar plate and inoculated into 10 ml L-broth+50 μg/ml kanamycin. The culture was grown overnight at 37° C. with shaking at 200 rpm. 1 ml of the overnight culture was inoculated into 3×100 ml in 250 ml conical flasks and grown at 37° C. with shaking at 200 rpm until the OD was ˜0.8 (log phase). Protein expression was induced by the addition of 100 mM IPTG to each culture to give a final concentration of 1 mM. The cultures were incubated at room temperature (RT) with shaking at 200 rpm for 6 hours. After incubation, the cultures were centrifuged at 5,000 rpm for 15 minutes to pellet the cells. The supernatants were discarded and the cell pellets were resuspended in 10 ml PBS, into which was previously dissolved one Complete protease cocktail tablet (Boehringer Mannheim). The cell suspensions were freeze-thawed by incubating at −20° C. overnight and thawing at RT. The lysed cells were sonicated using a probe sonicator for 5 minutes to denature the chromosomal DNA. The cell lysate was clarified by centrifugation at 10,000 g for 20 minutes and the soluble lysate decanted and stored at 4° C. The insoluble pellet was resuspended in 8M urea containing protease inhibitors, and incubated with gentle agitation at RT for 1 hour. This was to solubilise any ANTP-H10 that may have formed insoluble inclusion bodies within the periplasm (expression of scFv results in the formation of insoluble inclusion bodies that can be solubilised using urea). After incubation, the solution was centrifuged at 10,000 g for 20 minutes to pellet any remaining insoluble material. The supernate containing solubilised protein was decanted.

Purification and Re-Folding of ANTP-H10

Soluble and re-solubilised ANTP-H10 was dialysed overnight against phoshate buffer (10 mM Na₂HPO₄.2H₂O, 10 mM NaH₂PO₄.H₂O, 0.5 M NaCl, pH 7.4) and then purified using a His-Trap purification kit (Pharmacia Biotech) according to the manufacturer's instructions. The ANTP, scFv and c-myc genes were cloned in-frame with a 6-histidine tag that can be purified using a nickel chelate column. The His-Trap column (1 ml volume) was washed with 5 ml dH₂O to wash off the isopropanol storage buffer. The column was primed with 0.5 ml NiSO₄ (supplied in kit) and again washed with 5 ml dH₂O to remove any excess NiSO₄. The column was washed with 10 ml start buffer (supplied in kit) and the soluble ANTP-H10 sample was applied to the column. The column was again washed with 10 ml start buffer to wash off any unbound protein. Bound protein was eluted with 5 ml elution buffer (supplied in kit) and the eluted protein was collected in 5 ml fractions. The column was regenerated with 10 ml start buffer and the re-solubilised fraction was purified as described above. As can be seen in FIG. 5, the eluted ANTP-H10 protein is almost completely pure. Half the pooled sample was dialysed against arginine re-folding buffer and half against Tris-HCl, NaCl, Triton X-100 (TN) re-folding buffer.

Wash buffer after application of each sample was collected for analysis to determine if the protein was binding to the column. Samples were stored at 4° C. until analysis by Western blot and ELISA.

Analysis of Samples by ELISA

The soluble and re-solubilised fractions were purified using a His-Trap purification kit. The soluble fraction was purified first and five 1 ml fractions were collected. The column was regenerated and the re-solubilised fraction was collected. Eluted samples, flow-through of the protein samples after application to the column and the wash buffer after application of the samples were collected and analysed by ELISA to determine a) in which fractions the ANTP-H10 protein was eluted and b) if the protein was binding to the column.

Dynatech Immulon II 96 well microplates (Dynatech Laboratories) were coated with BPL-inactivated VEE TC83 at 10 μg/ml in carbonate-bicarbonate coating buffer, pH 9.6 (Sigma Chemical Co.) overnight at 4° C. (100 μl/well). The plates were washed ×3 with PBST and the wells of the plates were blocked with 100 μl/well Blotto at RT for 1 hour. The plates were emptied and 100 μl Blotto was added to all wells of the plates except row B. 150 μl Blotto was added to these wells, together with 50 μl each sample into triplicate wells of row B (¼ dilution). The diluted samples were serially diluted two-fold down the plates. The plates were incubated at RT for 2 hours. The plates were washed ×3 with PBST and 100 μl 1/500 dilution anti c-myc MAb 9E10, diluted in Blotto, was added to all wells and the plates were incubated at RT for 1 hour. The plates were then washed ×3 with PBST and 100 μl/well 1/4000 dilution anti-mouse HRPO conjugate in Blotto was added to all wells. The plates were incubated at RT for 1 hour and then washed ×5 with PBST. 50 μl 5,5′,3,3′tetramethyl benzidine (TMB, Sigma Chemical Co.) diluted in phosphate-citrate buffer containing urea-H₂O₂ (PCB, Sigma Chemical Co.) was added to all wells. The plates were incubated at RT for 30 minutes and the reactions were stopped by the addition of 25 μl 2M H₂SO₄. The plates were read at 450 nm using a Titertek Multiskan MCC plate reader. FIG. 4 shows the result of the ELISA.

The graph shows the absorbance values for the above samples from an ELISA assay at a dilution of 1/10 of each sample. As can be seen, the ANTP-H10 protein elutes mainly in the first three elution fractions. Some ANTP-H10 protein can be seen in the flow-though after the fraction was applied to the column. This indicates that some protein did not bind to the column, probably due to saturation of the 6-His binding sites on the column. It can be seen from the result that most of the protein is binding to the His-Trap column and that the ANTP-H10 protein is eluted in the first three elution samples. The first three fractions of the soluble and re-solubilised protein samples were pooled together. The pooled eluted samples, flow-through and wash buffer were analysed by SDS-PAGE and Western blot to determine the purity of the purified ANTP-H10.

Analysis of Samples by Western Blot

Soluble, re-solubilised and insoluble fractions were analysed by Western blot to determine in which fraction the ANTP-H10 protein was located. Two 12.5% SDS-PAGE gel were set up according to the method of Laemmli (Laemmli, 1970 Nature, 277, 680). 10 μl each sample was diluted in 10 μl 2×Laemmli sample buffer (Sigma Chemical Co.) and boiled for 5 minutes. 10 μl each sample was loaded onto each gel together with 5 μl pre-stained broad range molecular weight markers (Bio-Rad). The gels were run at 200V for 1 hour. One gel was stained with Coomassie blue stain (10% glacial acetic acid, 10% methanol, 0.1% Coomassie blue stain in dH₂O) for 4 hours, followed by de-staining (10% glacial acetic acid, 40% methanol in dH₂O) overnight. The other gel was blotted onto a PVDF membrane as follows. 1 PVDF membrane (Millipore) and 4 sheets of blotting paper were cut to the size of the gel and soaked in transfer buffer (14.4 g glycine, 3.03 g Tris, 100 ml methanol made up to 1 litre in dH₂O). Two pieces of soaked filter paper was placed onto the cathode of a Biometra blotting apparatus and the soaked PVDF membrane placed on top. The gel was placed onto the PVDF membrane and the remaining filter paper placed on top. The anode was fixed into place and a current of 0.2 amps ran through the sandwich for 1 hour. The blotted PVDF membrane was blocked with Blotto (1% skimmed milk powder in PBS+0.1% Tween 20) at RT for 1 hour and 10 ml 1/500 dilution anti-myc MAb 9E10 (Sigma Chemical Co.) in Blotto was added to the blot. The blot was incubated at RT with shaking for 1 hour. The blot was washed ×2 for 5 minutes in PBST and 10 ml 1/1000 dilution anti-mouse HRPO conjugate (Sigma Chemical Co.) in Blotto was added to the blot. The blot was incubated at RT with shaking for 1 hour. The blot was washed for 15 minutes in PBST, followed by 4×5 minute washes in PBST. 10 ml DAB substrate (Pierce and Warriner, Chester) was added to the blot and the blot developed for 20 minutes at RT. The blot was washed in dH₂O to stop the reaction.

FIG. 3 shows the results of the blot. As can be seen from the blot, some protein is excreted into the culture medium. However, most protein aggregates as insoluble inclusion bodies within the periplasm. The insoluble protein can be re-solubilised using 8M urea, however, a proportion of protein remains in the insoluble fraction (lanes 4 and 5). This protein is probably mis-folded and non-conformational, which is probably due to point mutations within the protein sequences.

The ANTP-H10 protein is shown by the arrow, the molecular weight of the ANTP-H10 protein is ˜32 kDa. Equal amounts of the protein are present in the soluble and re-solubilised fractions, showing that the expression of ANTP-H10 is overwhelming the re-folding pathways in the periplasm. No ANTP-H10 is visible in the remaining insoluble fraction. The blot also shows some protein breakdown products that are produced by the proteolytic cleavage of the protein by bacterial proteases. In order to avoid the loss of protein by proteolytic cleavage, protease inhibitors (in this case Boehringer Mannheim Complete protease inhibitors) are suitably added to the protein samples after expression and at all steps during protein purification.

Re-Folding of ANTP-H10 and Translocation into Vero Cells

Eluted fractions containing ANTP-H10 as analysed by ELISA were pooled. Half the pooled ANTP-H10 was refolded by overnight dialysis against arginine re-folding buffer (0.1M Tris-HCl, 0.4M L-arginine, pH 8.0), which optimally re-folds the scFv fragment. The remaining half of the pooled ANTP-H10 was re-folded by overnight dialysis against a re-folding buffer shown to optimally re-fold the ANTP protein. This buffer is 0.1M Tris-HCl, 0.1M NaCl, 0.1% Triton X-100, pH 8.0. Dialysed protein was collected, divided into 200 μl aliquots and stored at −20° C.

Glass-bottomed cell culture dishes (Wellco) were seeded with Vero cells at 1×10⁵ cells/ml and incubated overnight at 37° C. in a humidified CO₂ incubator. Vero cell culture medium was made up and 5 ml 200 mM calcium chloride was added to the medium to give a final concentration of 2 mM. 100 μl ANTP-H10 in the different re-folding buffers were diluted in 1900 μl medium and added to the cell culture dishes. The dishes were incubated at 37° C. in a humidified CO₂ incubator. Two dishes received cell culture medium with 2 mM Ca²⁺, but no ANTP-H10 as negative controls. After 1, 2, 3 and 4 hour's incubation, one dish containing ANTP-H10 in each re-folding buffer was fixed in 4% paraformaldehyde by removing the cell culture medium, washing the cell monolayers twice in Staining Buffer (PBS+2% FCS), and adding 1 ml 4% paraformaldehyde (Merck). The dishes were fixed for 20 minutes. After fixing, the cell sheets were washed once with staining buffer and once in permeabilization buffer (PBS+0.1% saponin+2% FCS). 1 ml/plate 1/500 dilution anti c-myc MAb 9E10 in staining buffer was added to each plate and the plates incubated on ice in the dark for 30 minutes. The cell sheets were washed twice in permeabilization buffer. 1 ml/plate 1/40 dilution anti-mouse FITC conjugate (Sigma Chemical Co.) was added to each dish and the plates were incubated for 30 minutes at 4° C. The cell sheets were washed once in permeabilization buffer and once in staining buffer. The cell sheets were examined by confocal laser microscopy for the presence of the ANTP-H10 protein inside the cells.

Glass-bottomed cell culture dishes, seeded with Vero cells, were used in the translocation experiments. Each re-folded ANTP-H10 sample was diluted in GMEM cell culture medium containing 2mM CaCl₂ and 2 ml added to each dish. The dishes were incubated at 37° C. for 1, 2, 3 and 4 hours, together with a control containing no ANTP-H10. The dishes were fixed and stained, and viewed using a confocal laser microscope. FIG. 6 shows some of the results. The ANTP-H10 protein re-folded in TN buffer (A) precipitated either on the surface of the cells or within the cells, although the precipitation is most probably on the surface of the cells. The arrow points to the precipitated protein. The ANTP-H10 protein re-folded in arginine buffer (B) has translocated into the cells and can be seen in the cytoplasm of the cells. The arrow points to a cell containing ANTP-H10 in the cytoplasm. The nucleus can be seen in the centre of the cell. No fluorescence was seen on the negative control slides indicating that the fluorescence seen on the other slides is due to the presence of the ANTP-H10 protein.

ANTP-H10 protein re-folded using arginine buffer can be seen within the cell cytoplasm after 4 hours incubation. ANTP-H10 protein re-folded using TN buffer has precipitated and it is not clear whether the protein is located within the cytoplasm as precipitates, or whether the precipitated protein has aggregated on the surface of the cells. No immunofluorescence is seen on the negative control cell sheets, indicating that the immunofluorescence seen on the cell sheets containing the ANTP-H10 protein is occurring from the protein.

The results indicate that ANTP-H10, when re-folded using arginine-containing buffer, can translocate into Vero cells, where they would be expected to produce an antiviral effect.

1. A protein conjugate comprising:
(i) a first region comprising a factor that permits translocation of a protein across a cell membrane; and
(ii) a second region comprising an single-chain antibody fragment which has affinity for a viral protein.

2. A protein conjugate according to claim 1 wherein the said first region comprises the homeodomain of antennapedia, or a functional fragment or homologue thereof.

3. A protein conjugate according to claim 1 or claim 2 wherein said viral protein is a protein of a flavivirus, an alphavirus, an enterovirus, an arboviruses, a retrovirus, a respiratory virus, a rhabdovirus, a herpes virus, human papilloma virus (HPV), an adenovirus, an adenavirus or a pox virus.

4. A protein conjugate according to any one of the preceding claims, wherein the virus is Flavivirus.

5. A protein conjugate according to claim 4, wherein the Flavivirus is hepatitis C virus, dengue virus or tick-borne encephalitis virus.

6. A protein conjugate according to any one of the preceding claims wherein the viral protein is a protein necessary for replication of virus.

7. A protein conjugate according to any one of the preceding claims wherein said viral protein is a non-structural viral protein.

8. A protein conjugate according to claim 7, wherein the single-chain antibody fragment has affinity for a Flavivirus non-structural protein, identified herein as any of NS1, NS2, NS3, NS4A, NS4B, NS5a and NS5B.

9. A protein conjugate according to claim 8 wherein the non-structural protein comprises an NS2 or NS3 protein of a flavivirus.

10. A protein conjugate according to claim 9, wherein the non-structural protein comprises an NS3 protein of a flavivirus.

11. A protein conjugate according to any one of claims 1 to 6 wherein said viral protein is a structural protein.

12. A protein conjugate according to claim 11 wherein the structural protein is an E1 or E2 protein of an alphavirus.

13. A protein conjugate according to any one of the preceding claims which further comprises a therapeutic agent.

14. A protein conjugate according to any one of the preceding claims which further comprises an intracellular localisation moiety.

15. A protein conjugate according to any one of the preceding claims in the form of a fusion protein.

16. A protein conjugate according to claim 15 wherein said first and second region and/or any therapeutic agent present and/or any intracellular localisation moiety are spaced by a spacer amino acid sequence.

17. A protein conjugate according to claim 16 wherein said spacer amino acid sequence includes a cleavage site of an intracellular enzyme.

18. A polynucleotide which encodes a protein conjugate according to any one of claims 15 to 17.

19. A vector which comprises a polynucleotide according to claim 18.

20. A cell which has been transformed with a vector according to claim 19 which is capable of expressing a protein conjugate according to any one of claims 15 to 19.

21. A cell according to claim 20 which comprises a gut-colonising organism.

22. A cell according to claim 21 which comprises an attenuated Salmonella.

23. A recombinant virus which has been transformed with a vector according to claim 19 which is capable of expressing a protein conjugate according to claim 15.

24. A recombinant virus according to claim 23 which is an attenuated virus.

25. A recombinant virus according to claim 24 which comprises an attenuated vaccinia virus.

26. A pharmaceutical composition comprising a protein conjugate according to any one of claims 1 to 17, a polynucleotide according to claim 18, a cell according to claim 20 or claim 21 or a recombinant virus according to any one of claims 23 to 25, in combination with a pharmaceutically acceptable carrier or diluent.

27. A pharmaceutical composition according to claim 26 which further comprises a therapeutic agent.

28. A composition according to claim 27, wherein the therapeutic agent is capable of inactivating the NS3 protein.

29. A composition according to claim 27 or claim 28, wherein the therapeutic agent is a serine protease inhibitor, a NTPase inhibitor or a helicase inhibitor.

30. A method for the preparation of a protein conjugate according to any of claims 1 to 17, comprising culturing a cell according to claim 20 and recovering the protein conjugate.

31. A method according to claim 30 wherein protein conjugate recovered is purified under non-denaturing conditions.

32. A method according to claim 30 or claim 31 wherein recovered protein conjugate is refolded prior to use.

33. A method according to any one of claims 30 to 32 which is effected in the presence of a protease inhibitor.

32. A protein conjugate substantially as hereinbefore described.
